(19) United States
(12) Patent Application Publication
(10) Pub. No.: US 2010/0003271 A1
(43) Pub. Date: Jan. 7, 2010

(54) NITRIC OXIDE INCREASES SWITCHING OF T CELLS INTO T REGULATORY CELLS

(75) Inventor: Stanislaw M. Stepkowski, Pearland, TX (US)

Correspondence Address: CONLEY ROSE, P.C.;David A. Rose, P. O. BOX 3267, HOUSTON, TX, 77253-3267, US

(73) Assignee: BOARD OF REGENTS OF THE UNIVERSITY OF TEXAS SYSTEM, AUSTIN, TX (US)

(21) Appl. No.: 12/518,982

(22) PCT Filed: Dec. 17, 2007
(86) PCT No.: PCT/US07/87727

§ 371 Date: Jul. 27, 2009

Related U.S. Application Data
(60) Provisional application No. 60/870,182, filed on Dec. 15, 2006.

(51) Int. Cl. A61K 39/00; C12N 5/02

(52) U.S. Cl. 424/184.1; 435/378

(57) ABSTRACT

An ex vivo method of expanding a population of regulatory T-cells includes culturing a starting population of cells containing CD4⁺CD25⁻ T-cells in a growth medium; introducing nitric oxide into the growth medium sufficient to potentiate switching of the CD4⁺CD25⁻ T-cells to CD4⁺CD25⁺ regulatory T-cells (Treg cells), whereby a subpopulation of Treg cells is produced; and allowing the Treg cells to proliferate in culture, to provide a final population of T-cells containing more Treg cells than were present in the original T-cells. The resulting expanded population of Treg cells are used to deter or decrease an undesired T-cell mediated immune response, e.g., allograft rejection, in a mammalian host by transplanting the Treg cells at a site of a potential or existing undesired immune response, whereby the undesired immune response is deterred or decreased.

### CROSS-REFERENCE TO RELATED APPLICATIONS

The present application claims priority under 35 U.S.C. § 365 to International Patent Application No. PCT/U.S. 2007/087727 filed on Dec. 17, 2007, which claims the benefit of U.S. Provisional Patent Application No. 60/870,182 filed Dec. 15, 2006. The disclosures of those applications are hereby incorporated herein by reference.

### STATEMENT REGARDING FEDERALLY SPONSORED RESEARCH OR DEVELOPMENT

This invention was made with government support under HL069723 awarded by the National Institutes of Health. The government has certain rights in the invention.

### BACKGROUND

T cells not only initiate the cascades of events leading to tissue damage (such as the autoimmune diseases and allograft rejection), but also regulate these destructive processes (Veldman C, Int Arch Allergy & Immunol 140:174, 2006; Akl A, Transpl Immunol 14:225, 2005). The natural CD4⁺CD25⁺FoxP3⁺ T regulatory (Treg) cells maintain self-tolerance and display critical suppressive activity inhibiting CD4⁺ T cell proliferation (Sakaguchi S, 2004; Gavin and Rudensky, 2003) and influencing highly the autoimmunity (Suri-Payer et al., 1998; Roncarolo, and Levings, 2000; Shevach A M, 2000), transplantation tolerance (Hall et al., 1998; Wood and Sakaguchi, 2003) as well as anti-tumor immunity (Peng et al., 2002; Gallimore and Sakaguchi, 2002) and anti-infectious responses (Belkaid et al., 2002; Suvas S et al., 2003). The Tregs suppress immune responses through direct cell-cell interactions as well as the release of inhibitory cytokines such as interleukin-10 (IL-10) and transforming growth factor-beta (TGF-beta) (Roncarolo et al., 2001; Shevach EM, 2000; Shevach et al., 2001). Currently, there are no methods to expand the number of Treg cells for in vivo use to block undesirable responses, protect from autoimmune diseases, and to inhibit allograft rejection process or even to induce transplantation tolerance.

The NO is produced at higher levels by the activated macrophages and other cells like dendritic cells and neutrophills. Interestingly, a dichotomous regulation between the NO and the T-cell priming responses has been noticed (Mukhopadhyay et al. 1999a). It was shown showed that the bruton's tyrosin kinase deficient mice (btk) producing lower amount of NO could mount stronger antigen-specific T cell proliferation as compared to the wild type control mice (CBA/J).

Further studies using the inducible NO synthase-2 inhibitor (aminoguanidine (AG)) strengthened the fact that T cell proliferation is negatively regulated by NO (Mukhopadhyay et al., 1999b). Since NO is a cytotoxic molecule, at certain concentrations it may affect T cell viability (Vig et al., 2004).

### SUMMARY

Nitric oxide increases switching of CD4⁺CD25⁻ cells into CD4⁺CD25⁺CD127⁻FoxP3⁺ T regulatory cells. In accordance with certain embodiments of the invention, an ex vivo method of expanding a population of regulatory T-cells is provided which comprises: culturing a starting population of cells containing CD4⁺CD25⁻ T-cells in a growth medium; introducing nitric oxide into the growth medium sufficient to potentiate switching of said CD4⁺CD25⁻ T-cells to CD4⁺CD25⁺ regulatory T-cells (Treg cells), whereby a subpopulation of Treg cells is produced; allowing said Treg cells to proliferate in culture, to provide a final population of T-cells containing a greater number of Treg cells than did said starting population of T-cells. In some embodiments, the method includes isolating the resulting expanded population of Treg cells. The starting population of T-cells may comprise peripheral blood lymphocytes, for example, and the growth medium may include PHA.

In some embodiments, the nitric oxide is introduced into the growth medium by addition to the medium of a nitric oxide producing agent selected from the group consisting of S-Nitroso-N-acetyl-DL-penicillamine (SNAP), sodium nitroprusside (SNP), 3-morphylynosydnonimine (SIN-1), naproxen (HCT-3012 [(S)-6-Methoxy-α-methyl-2-naphthaleneacetic acid 4-(Nitrooxy)butyl ester]), sodium nitroprusside, 2-2-(hydroxynitrosohydrazino)bis-ethamine (NOC-18) and arginine, or another suitable nitric oxide producing agent. The concentration of nitric oxide, after introduction of the NO producing agent into the medium, is in the range of 10 to 100 micromolar, in some embodiments.

Also provided in accordance with certain embodiments is an in vitro method of inhibiting proliferation of immune responsive T-cells. This method comprises culturing a population of immune responsive T-cells in the presence of an immunogenic agent and in the presence of a population of Treg cells that are prepared in accordance with a Treg cell population expansion method described above.

Further provided in accordance with embodiments of the invention is a method of deterring or decreasing an undesired T-cell mediated immune response, which comprises obtaining an isolated population of Treg cells produced as described above; and transplanting the Treg cells into a mammalian host at the site of a potential or existing undesired immune response, whereby the undesired T-cell mediated immune response at said site is deterred or decreased. In certain embodiments, transplanting the Treg cells into the host at the site of a potential or existing undesired immune response blocks the undesired T-cell mediated immune response at that site. In some embodiments, the site that has an existing undesired immune response, or is subject to a potential undesired immune response, comprises an allograft. In some embodiments, the population of transplanted Treg cells at the transplantation site is sufficient to enhance acceptance of the allograft. In some embodiments, the method includes expanding the population of the transplanted Treg cells at the transplantation site sufficient to inhibit chronic allograft rejection in the host. In some embodiments, the method of deterring or decreasing an undesired T-cell mediated immune response comprises expanding the population of transplanted Treg cells at the transplantation site sufficient to induce transplantation tolerance in the host. For example, in some embodiments the host suffers from an autoimmune disease and the transplantation site includes an existing or potential target site for attack by autoimmune T-cells. These and other embodiments, features and advantages will be apparent in the accompanying drawings and in the following description.

### DESCRIPTION OF THE DRAWINGS

FIGS. 1A-C. Impact of NO concentrations on viability of T cells. (FIG. 1A) PBLs were stimulated with PHA and cultured in the presence of different SNAP concentrations (0-1000 mM). Cell viability was evaluated by an MTT assay; (FIG. 1B) PBLs were stimulated with PHA and cultured in the presence of different SNAP concentrations (0-100 mM). Cell viability was evaluated by an APO-BrdU assay. (FIG. 1C) Supernates were collected within 24 hrs of PBL culture in the presence of SNAP and nitrites concentrations measured by a standard method.

FIGS. 2A-B. NO increases generation of CD4⁺CD25^{high}CD127⁻ Treg cells. PBLs were either non-activated and cultured with different SNAP concentrations (FIG. 2A-1), or were activated with PHA and cultured with DMSO alone (FIG. 2A-2) or with different concentrations of DMSO/SNAP (FIG. 2A-3). Following 5-day culture cells were labeled with CD4-perCP, CD25-APC and CD127-PE to select CD4⁺CD25^{high}CD¹²⁷⁻ population by FACS analysis. (FIG. 2B) Three independent experiments were performed to calculate mean±SD values for identical cultures with DMSO alone or with DMSO and different SNAP concentrations. Statistical significance was calculated using Student-t test and p<0.05 was considered as significant.

FIGS. 3A-B. NO induces generation of Foxp3-expressing CD4⁺CD25^{high}CD127⁻ Treg population. PBLs activated with PHA and cultured with DMSO alone (FIG. 3A) or cultured with different SNAP concentrations (FIG. 3B). Following 5-day culture cells were labeled with CD4-perCP, CD25-APC and CD127-PE to select CD4⁺CD25^{high}CD127⁻ population, which was tested for intracellular expression of Foxp3 by FACS analysis.

FIGS. 4A-B. CD₄⁺CD25^{high} Treg cells inhibit proliferation of PBLs in response to alloantigen or PHA. PBLs were stimulated with irradiated HLA incompatible leukocytes for 5 days. Following culture CD4⁺CD25^{high} and CD4⁺CD25^{low} cells were isolated using cell sorter. These populations were added at different ratios to the same responder primary MLC stimulated with the same irradiated HLA incompatible leukocytes (FIG. 4A) or with PHA (FIG. 4B).

FIGS. 5A-D. Alloantigen-specific Treg cells blocks rejection of skin allografts. (FIG. 5A) 60×10⁶ PBLs were cultured with irradiated HLA non-compatible PBLs for 5 days and the number of CD4⁺CD25^{high} population was evaluated by FACS analysis. (FIG. 5B) 20×10⁶ Balb/c spleen mononuclear cells were cultured for 5 days with irradiated C57BL/6 splenoctes without or with 100 μM SNAP o; CD4⁺CD25^{high} population was purified by FACS and injected i.v. (2-5×10 per mouse) to Balb/c SCID recipients of C57BL/6 skin allografts; within 7 days the same recipients were injected i.v. with 1×10⁵ CD4⁺CD45⁺ cells isolated from naive Balb/c mice. Control recipients were injected only with 1×10⁵ CD4⁺CD45⁺ cells. Skin allograft survival was evaluated daily and 50% graft damage was considered as rejection. FIG. 5C Balb/c T+B6 cells. FIG. 5D Balb/c T+B6 cells+SNAP.

FIGS. 6A-C. NO increases switching of CD4⁺CD25⁻ cells into CD4⁺CD25^{high}CD127⁻ Treg population. (FIG. 6A) Isolated human CD4⁺CD25⁻ cells labeled with carboxyfluorescein diacetate succinimidyl ester (CFSE) and co-cultured with naive CD4⁺CD25⁺ cells stimulated with irradiated HLA-mismatched cells in the presence of DMSO alone or DMSO and 30, 50 or 100 μM SNAP. Following 5 day-culture, cells were labeled with CD24-APC and CD127-PE and CFSE⁺CD25^{high}CD127⁻ cells were identified by FACS. Results in right corners indicate at % of selected population in one experiment. (FIG. 6B) Three independent experiments were performed to calculate mean±SD values for identical cultures with DMSO alone or with DMSO and different SNAP concentrations (FIG. 6C). Statistical significance was calculated using Student-t test and p<0.05 was considered as significant.

### DETAILED DESCRIPTION

NO at low concentrations can also act as an immunomodulator to regulate T cell-mediated immune response. Our studies revealed that NO at low concentrations may inhibit T cell proliferation by increasing the generation of Treg cells. It was also documented that NO potentiates switching of CD4⁺CD²⁵⁻ cells into CD4⁺CD25⁺ Treg cells.

Natural CD4⁺CD25⁺ regulatory T (Treg) cells are pivotal in self-tolerance to prevent autoimmune diseases as well as in maintaining tolerance to allografts. It was examined whether nitric oxide (NO) regulates function of Treg cells. Our results reveal that low NO concentrations (10-100 μM nitrite), released from S-Nitroso-N-acetyl-DL-penicillamine (SNAP) or sodium nitroprusside (SNP), increased 3-fold generation of CD4⁺CD25⁺CD127⁻FoxP3⁺ Treg cells, which inhibited proliferation of T cells in response to phytohemagglutinin (PHA) or alloantigens. In the presence of Treg cells, SNAP-released NO elevated switching of CD4⁺CD25⁻ cells into CD4⁺CD25⁺CD127⁻ FoxP3+ T regulatory (Tr1) cells. When tested in vivo, SNAP-induced Treg/Tr1 cells protected long-term survivals of skin allografts otherwise rejected by CD4⁺CD45⁺ cells in SCID mice. Thus, NO modulates function of Treg and Tr1 cells.

### Materials and Methods

### Reagents and Antibodies

The RPMI 1640 medium (Hyclone, Logan, Utah) was supplemented with 2 mM L-Glutamine, 1% MEM vitamin solution, 0.01 M HEPES buffer, 2×10⁻⁵ M 2-mercaptoethanol, 1% penicillin-streptomycin (all from GIBCO BRL, Invitrogen, Carlsbad, Calif.) and 10% fetal calf serum (FCS; Hyclone) (RPMI-10). The anti-human CD25-Fluorescein isothiocyanate (FITC) antibody (Ab) (cat no. 555431, clone M-A251, mouse IgGI), anti-human CD25-allophycocyanin (APC) Ab (Cat No 555434, clone M-A251, mouse IgGI), anti-human CD4-phycoerythrin (PE) Ab (cat no 555347, clone RPA-T4, mouse IgGI), anti-human CD4 peridin chlorophyll protein (PerCP) Ab, anti-human CD127-PE Ab (cat no 557938, clone hIL-7R-M21, mouse IgG1) were all purchased from BD PharMingen (San Diego, Calif.). S-Nitroso-N-acetyl-DL-penicillamine (SNAP), sodium nitroprusside (SNP) and dimethyl sulfoxide (DMSO) were purchased from Sigma-Aldrich, (St. Louis, Mo.). As NO generator, SNAP was dissolved in DMSO (1 M stock solution) and kept frozen at 20° C. until used; the same DMSO volumes were always added to control cultures. In some experiments, another NO generator SNP dissolved in water was added.

### Purification of Human T Cells and Various CD4 T Cell Subsets

Peripheral blood lymphocytes (PBLs) were isolated from a buffy coat from blood of healthy volunteers or from blood bank. T cells (95% purity) were obtained by T cell negative isolation kit (InVitrogen; Cat No: 113.11). CD4⁺CD25^{high}, CD4⁺CD²⁵^{intermediate}, or CD4⁺CD25^{negative} cells were isolated by cell sorting on FACSAria (Becton Dickinson, San Diego, Calif.) using anti-human CD4-FITC and anti-human CD25-PE Abs. The cells were immediately used after isolation.

### Identification of CD4⁺CD25^{high} CD127^{low} T Cell Population

PBLs or purified T cells were either left untreated or treated with 10 μg/ml phytohemagglutanin (PHA, Sigma-Aldrich) at 1×10⁶ cells/2 mL in 24-well plates in the absence or presence of various concentrations of SNAP-DMSO, DMSO or SNP and cultured in RPMI-10 for 5 days. Cells were then harvested and stained with anti-human CD4-PerCP, anti-human CD25-APC and anti-human CD127-PE Abs. Flow cytometry analysis measured CD4⁺CD25^{high} CD127^{low} versus CD4⁺CD25^{high}CD127^{high} populations using FACSAria with Diva program or FACSCalibur with CellQuest software (BD Pharmingen, San Diego, Calif.).

### Intracellular FoxP3 Staining

Foxp3 intracellular staining was carried out using the FITC anti-human Foxp3 staining kit (BDBiosciences, San Diego, Calif.; Cat no 71-5776-40). In brief, PBLs from various groups were incubated with anti-human CD4-PerCP, anti-human CD25-APC and anti-human CD127-PE Abs for 20 minutes at room temperature for surface staining of CD4, CD25 and CD127 molecules, respectively. Cells were then washed, and fixed with Fix/Perm solution (30 minutes at 4° C.) and permeabilized with permeabilization buffer followed by blocking with rat serum (15 minutes at 4° C.). Cells were then stained with FITC-conjugated anti-FoxP3 or isotype control Ab (30 minutes at 4° C.). Cells were analyzed by flow cytometry for intracellular FoxP3 levels in CD4⁺CD25^{high}/CD127^{low} versus CD4⁺CD25^{high}/CD127^{high} cells on a FACSAria.

MTT (3-(4,5-Dimethyl Thiazol-2-yl)-2,5-Diphenyl Tetrazolium Bromide) assay

PBLs (3×10⁵/well/200 μl) were left untreated or treated with PHA in the presence of various concentrations of SNAP for 24 hours, and 5 mg/ml MTT was added to triplicate samples for 4 hours. Cells were lysed overnight with 100 gl lysing buffer (Mukhopadhyay et al., 1999b) and the absorbance was determined at 570 nm (each point represents mean value of 3 wells).

### APO-BrdU Assay for Apoptosis

An APO-BrdU™ kit (Phoenix Flow Systems, San Diego, Calif.) was used to measure apoptotic cells. PBLs (1×10⁶) were either left untreated or treated with various SNAP concentrations for 24 hours. Harvested cells (1×10⁶) were fixed in 1% paraformaldehyde (15 minutes at 4° C.) and resuspended in 50 μl of DNA labeling solution containing 5-bromo-deoxyuridine triphosphate (Br-dUTP) and terminal deoxynucleotidyl transferase (TdT) enzyme (60 minutes at 37° C.). Cells were rinsed and resuspended in 100 μl of anti-BrdU-FITC monoclonal Ab (30 minutes at room temperature). Cell suspensions were mixed with 0.9 ml PBS containing 2 μg/ml propidium iodide (Sigma-Aldrich) and 50 μg/ml RNase A and analyzed immediately by flow cytometry.

### Nitrite Estimation

The accumulated nitrite (from NO in SNAP/DMSO, DMSO or SNP cultures) was measured by Griess method (Mukhopadhyay et al., 2004). The 96-well plates were filled with sample and equal volume of Griess reagent (1% sulfanilamide and 0.1% naphthylethylenediamine (1:1) in 2.5% orthophosphoric acid). The plates were read at 550 nm absorbance and nitrite concentrations were calculated based on a standard curve from a prepared standard solution of sodium nitrite.

Allogeneic Restimulation Assays to Study T cell Effector-Suppressor Function

Alloactivation was set up by co-culturing responder PBLs (1-2×10⁶ cells/ml) in a total volume of 35 ml of RPMI-10 with fully HLA mismatched gamma-irradiated (30 Gy) stimulators (1:1 ratio) in the presence of SNAP/DMSO. After 5 days, cells were washed and stained with anti-CD4-FITC and anti-CD25-PE Abs to isolate CD4+CD25^{high} and CD4+CD25^{intermediate} populations by FACS sorting. These cell populations were then added to fresh syngeneic PBLs (1×10⁵) at different responder/regulator cell ratios (1/0, 1/0.005, 1/0.01, 1/0.05, 1/0.1, 1/0.5, and 1/1) with the same HLA-mismatched irradiated stimulators (1×10⁵). Cells cultured in a 200 μl volume of RPMI-10 in 96-well round-bottomed plates for 5 days and pulsed with 1 μCi of [³H]-thymidine for last 16 hours before harvesting. Cells were collected onto glass filters using an automated multi-sample harvester and the amount of [³H]-thymidine incorporation was measured with a scintillation counter (Packard, Meridan, Conn.). Proliferative responses are expressed as the mean [³H]-thymidine incorporation (counts per minute [cpm]) of triplicate wells±SD. In identical experiments, PBLs were activated with PHA (10 μg/ml) to examine the ability of NO-induced CD4+CD25^{high} population to suppress proliferation of alloantigen-specific MLC.

### Skin Transplant Model

Naïve SCID mice with a BALB/c background served as recipients and C57BL/6 mice as skin donors. Spleens from Balb/c mice were lysed of red blood cells and splenocytes were enriched for T cells using mouse T Cell Negative Isolation Kit (Invitrogen). These T cells were cultured with irradiated C57BL/6 spleen cells in the presence of 100 μM SNAP. After 5 days, cells were harvested and stained with anti-mouse CD4-FITC and anti-mouse CD25-PE Abs (BD PharMingen) to purify CD4+CD25^{high} cells by sorting (FACSAria, Beckton Dickinson). Pure CD4+CD25^{high} cells were injected i.v. in SCID mice with C57BL/6 skin allografts; grafts were ˜2×2 cm. Within 7 days, the same recipients received 1×10⁶ CD4+CD45RA^{high} cells purified by cell sorter and grafts were assessed daily for signs of rejection; 50% of the graft damage was considered as rejection.

### CFSE Labeling and T Cell Co-Culture Assay

The CD4⁺CD25^{negative} and CD4⁺CD25^{high} T cell populations were isolated from T cells by cell sorting (FACSAria). Next, CD4⁺CD25^{negative} cells were labeled with carboxyfluorescein diacetate succinimidyl ester (CFDA-SE; cat no C1157, Invitrogen) as described elsewhere (Hans et al., 2005). A stock solution of CFSE (5 mM) was diluted with PBS and added to the CD4⁺CD25^{negative} cells at final concentration of 1 gM per 1×10⁶ cells (20 minutes at 37° C.). After three washings, CFSE-labeled CD4⁺CD25^{negative} cells (1×10⁶) were co-cultured without or with CD4⁺CD25^{high} cells at 10:1 ratio without or with titrated SNAP or DMSO and 6 hours later cells were activated with PHA (10 μg/ml). Following 5 days cells were harvested and stained with anti-human CD25-APC and anti-human CD127-PE Abs to measure transformation of CD⁴+CD25^{negative} cells to CD25⁺CD127^{negative} cells by FACS. The same cells were added to a primary syngeneic MLC at responder/regulator cell ratios (1/0, 1/0.25, 1/0.5, and 1/1). Cells were cultured for 5 days and the [³H]-thymidine incorporation was measured as described above.

### Statistics

A paired, 2-tailed Student t test was used to determine the statistical significance of differences between proliferative responses: p<0.05 were considered significant.

### Results

### NO Increases CD4⁺CD25^{high}CD127⁻ Treg Cells in Activated PBLs Culture

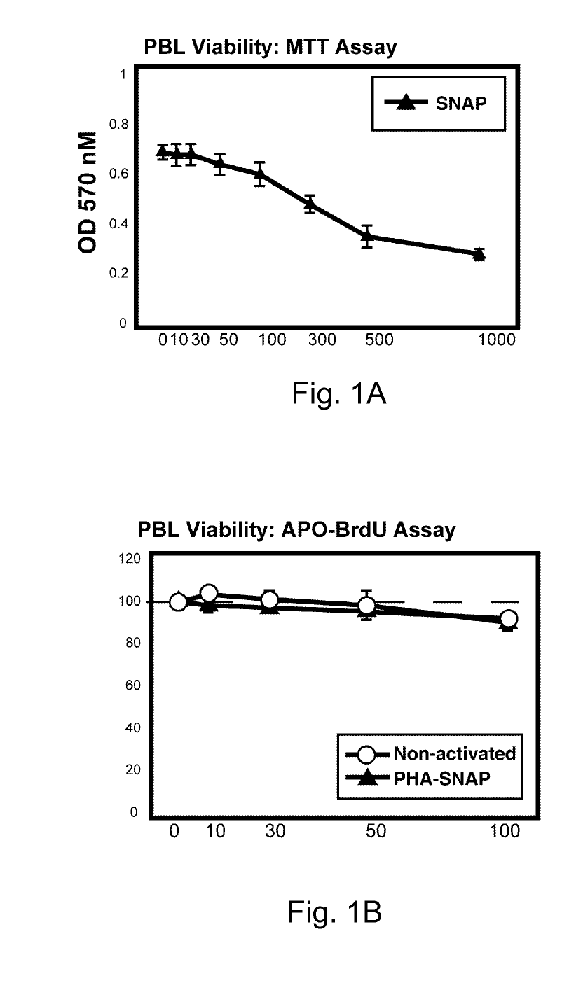

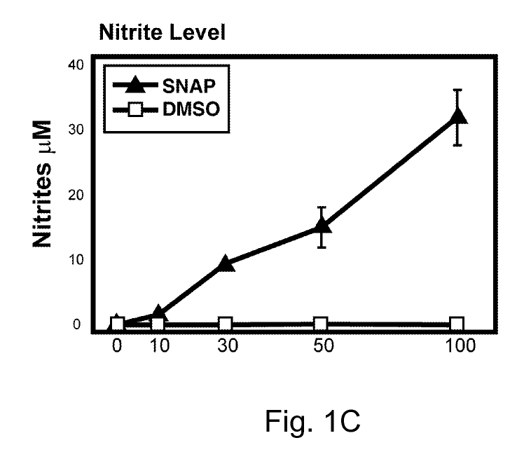

Previous studies showed that S-nitroso-N-acetylpenicillamine (SNAP) or sodium nitroprusside (SNP) serve as chemical source of NO (Hogan et al., 1992, Mukhopadhyay et al., 1999b). Cell viability assay (with 3-(4,5-dimethyl thiazol-2-yl)-2,5-diphenyl tetrazolium bromide; MTT) revealed that SNAP-released NO was significantly cytotoxic to peripheral blood lymphocytes (PBLs) only when increased to 300 μM and above (FIG. 1A). In fact, the 5-bromo-deoxyuridine triphosphate (APO-BrdU) apoptosis assay showed that SNAP-released NO concentrations between 10 to 100 μM produced less than 10% apoptotic cells among PHA-activated T cells in 5-day cultures (FIG. 1B). At the same time SNAP-released NO to the medium in a dose-dependent fashion, as shown by the elevated levels of nitrites (FIG. 1C).

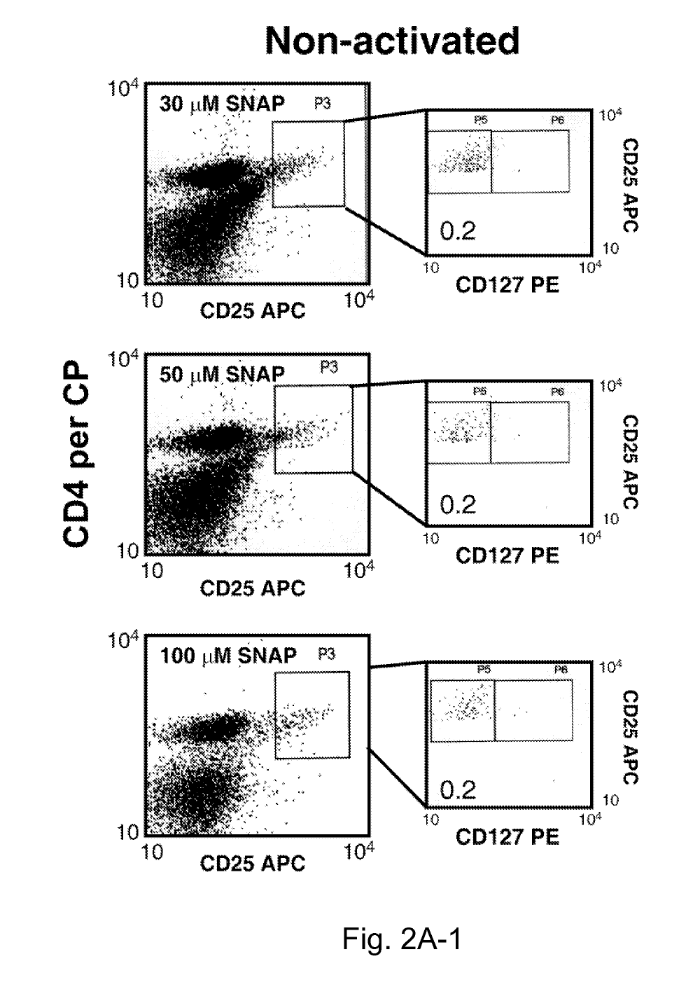

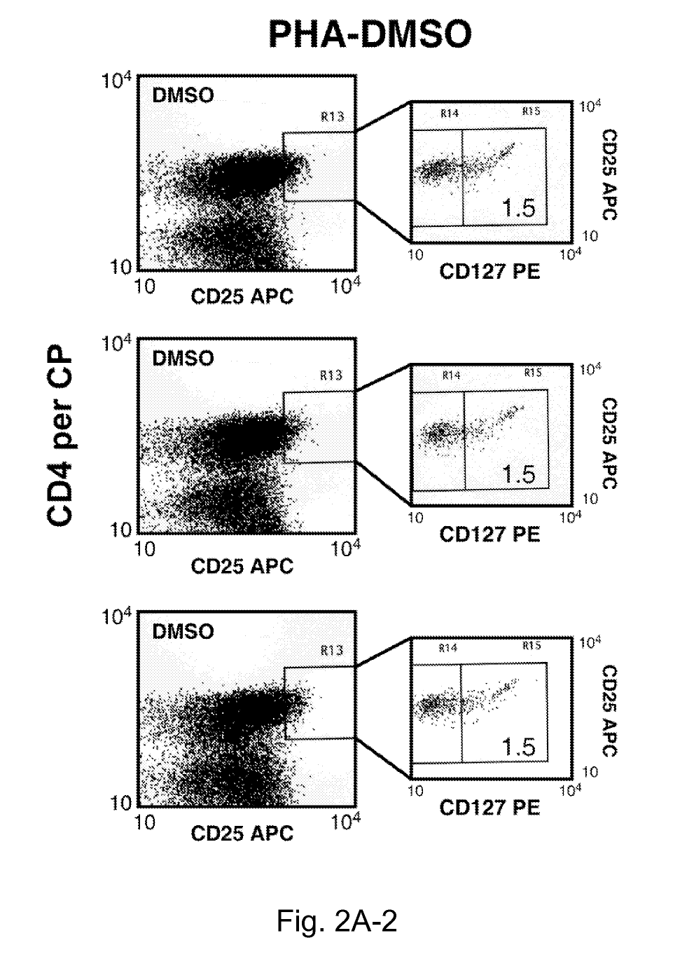

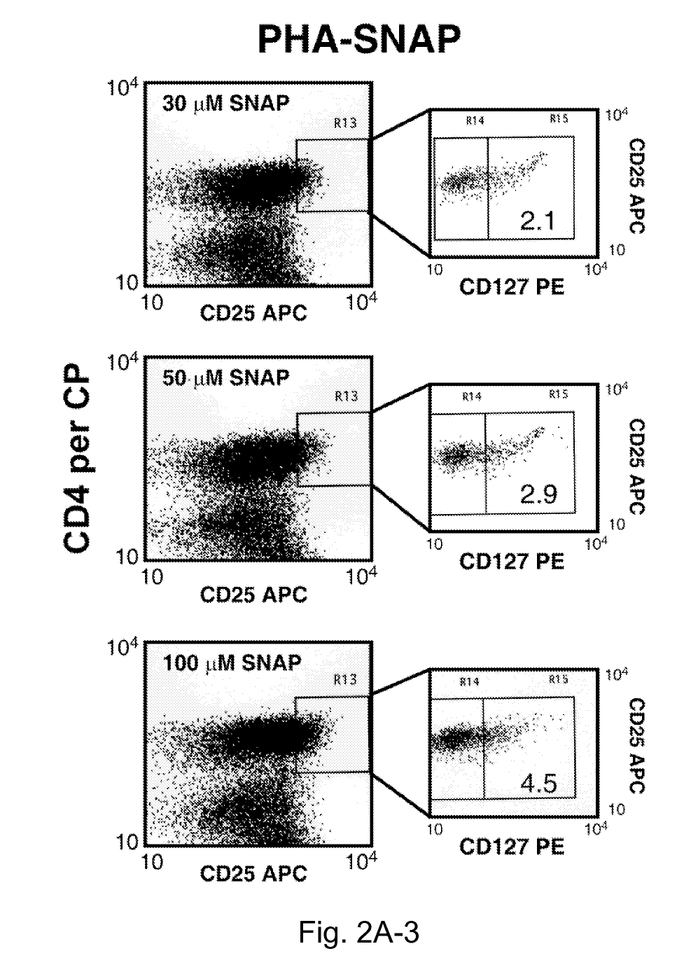

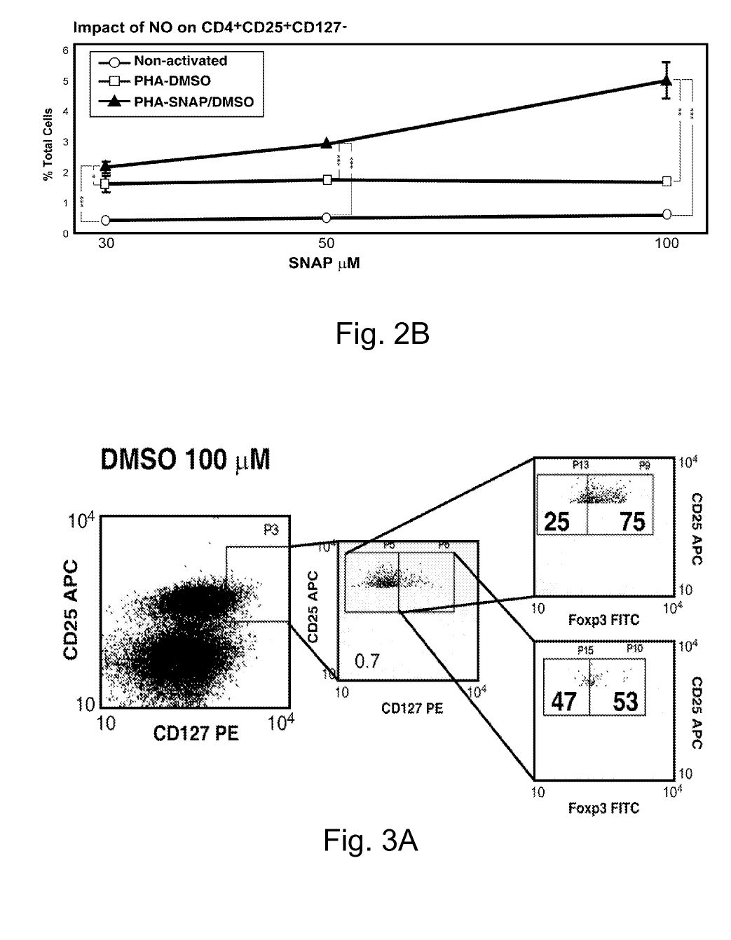

The non-activated Treg cells are characterized by higher membrane expression of IL-2Rα (CD25) and elevated intracellular expression of Foxp3 (Sakaguchi S, 2004). Since conventionally activated CD4⁺ T cells also express higher levels of CD25, identification of Treg cells must rely on different markers. Recently, lack of IL-7R has been confirmed as a potent phenotypic marker for CD4⁺CD25⁺ Treg for population with potent inhibitory functions (Seddiki et al., 2006). Therefore, the present experiments examined whether SNAP-released NO may affect generation of CD4⁺CD25⁺CD127⁻ Treg cells (FIGS. 2A-B). The non-activated PBLs exposed to different SNAP concentrations (FIG. 2A-1 top, middle and bottom panels) were compared with PHA-activated PBLs cultured for 5 days without (FIG. 2A-2, top, middle and bottom panels) or with different SNAP concentrations (FIG. 2A, top, middle and bottom panels). After culture, FACS was used to first select CD4⁺CD25^{high} population and then to examine it for the expression of CD127 to identify CD4⁺CD25⁺CD127⁻ Treg cells (FIGS. 2A-1, 2A-2 and 2A-3). Comparison of these populations showed that exposure to NO of PHA-activated PBLs significantly increased levels of CD4⁺CD25⁺CD127⁻ Treg cells (FIG. 2B). Since NO did not affect the number of CD4⁺CD25⁺CD127⁻ Treg cells among non-activated PBLs, activation step was required. Furthermore, elevated Treg numbers were also generated by another NO-generator, SNP, confirming that this phenomenon is caused by increased NO concentrations (not shown).

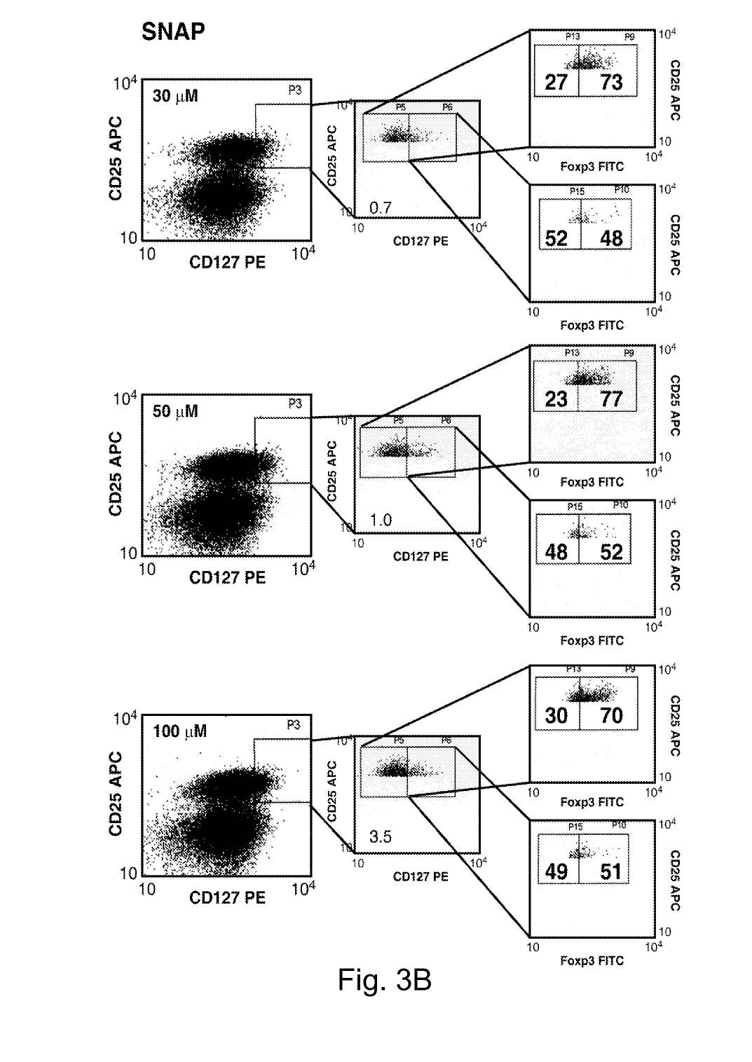

The master regulator Foxp3 expression inversely correlated with CD127 expression and in vitro suppression by CD4⁺CD25⁺CD127⁻ Treg cells (Seddiki et al., 2006). PHA-activated PBLs cultured without (FIG. 3A) or with SNAP (FIG. 3B) for 5 days confirmed that NO levels elevated numbers of CD4⁺CD25⁺CD127⁻ Treg cells. Top panel: 30 μM SNAP; Middle panel: 50 μM SNAP; Bottom panel: 100 μM SNAP. Intracellular staining for Foxp3 revealed that similar majority of Foxp3⁺ cells were among CD4⁺CD25⁺CD127⁻ Treg cells in all experimental groups. Thus, NO induces generation of Foxp3-positive CD4⁺CD25⁺CD127⁻ Treg cells.

### NO-Induced Treg Cells Inhibit T Cell Proliferation

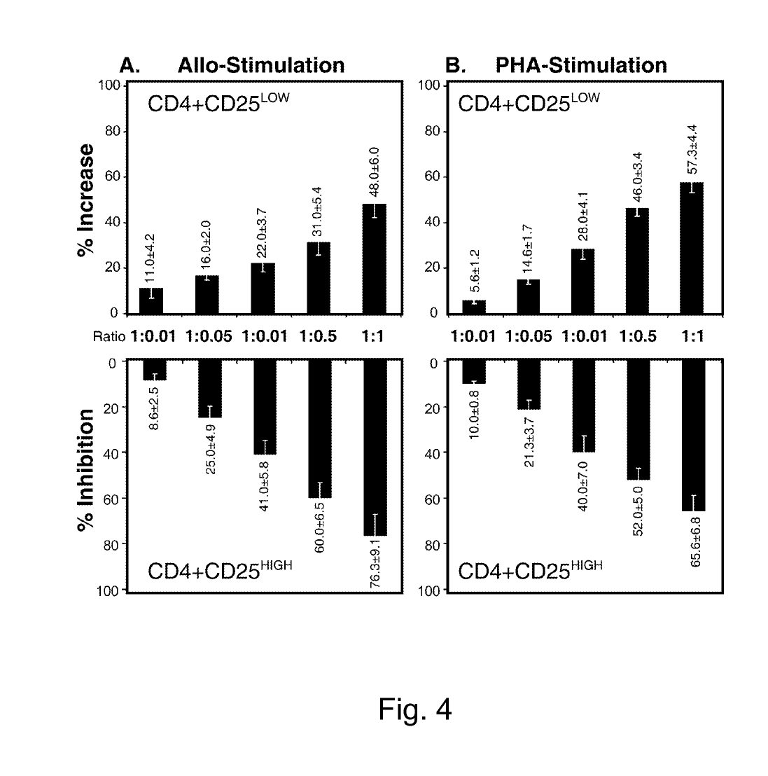

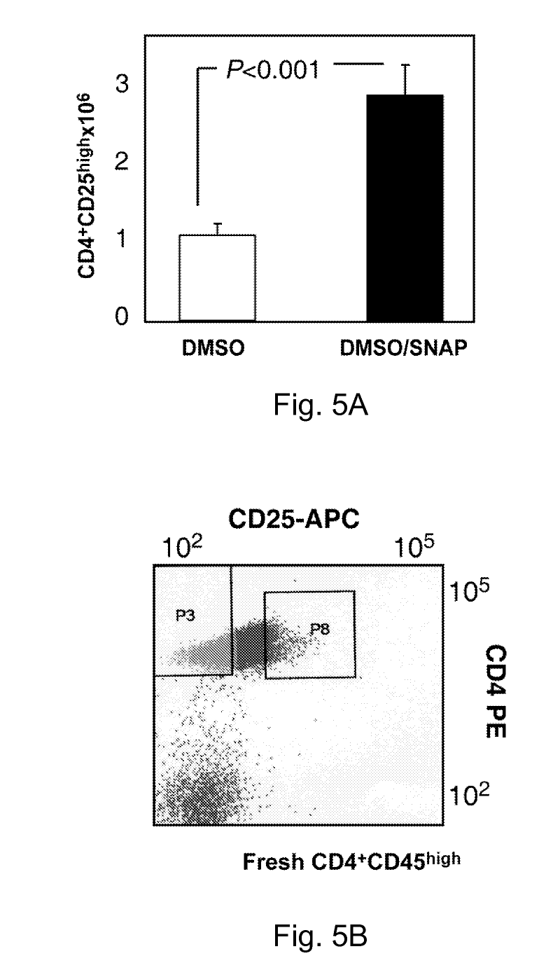

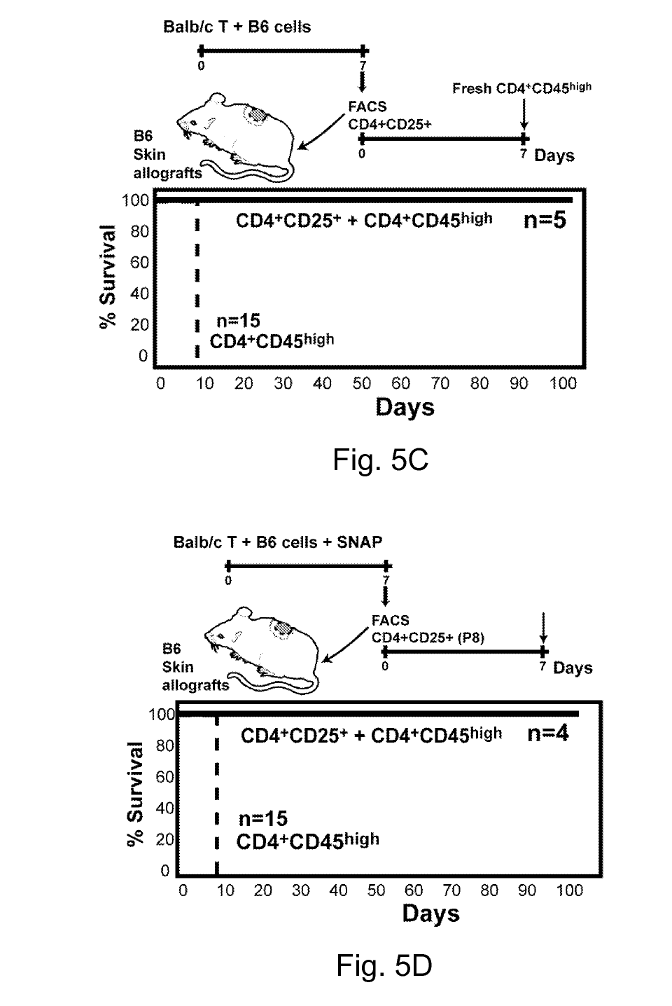

As a functional assay, it was examined whether NO-induced Treg cells may inhibit proliferation of T cells in response to alloantigen or PHA (FIGS. 4A-B). Population of Treg cells was always generated by culturing PBLs with HLA-mismatched irradiated stimulators for 5 days without or with 100 μM SNAP. Following culture, CD4⁺CD25^{low} and CD4⁺CD25^{high} cells were added at different responder/regulator ratios to the same primary MLC (FIG. 4A) or to the same responder stimulated with PHA (FIG. 4B). Top panels: CD4⁺CD25^{low}; bottom panels: CD4+CD25^{high}. The results demonstrated that CD4⁺CD25^{high} cells inhibited T cell proliferation in response to alloantigen or PHA. In contrast, CD4⁺CD25^{low} cells enhanced proliferative response, suggesting that this population may contain primed memory cells. The beneficial effect produced by adding of 100 μM SNAP to 60×10⁶ PBLs challenged with HLA-mismatched alloantigen was also calculated (FIG. 5A). While 5-day culture generated approximately 1×10⁶ Treg cells, almost 3-fold that number was generated in the presence of SNAP-released NO. Thus, NO significantly increases the number of functional CD4⁺CD²⁵^{high} Treg.

### NO-Induced Treg Cells Induces Transplantation Tolerance

An in vivo model was established to test whether NO-induced Treg cells may block allograft rejection or even induce permanent acceptance of allografts. Spleen Balb/c cells were cultured for 5 days with irradiated C57BL/6 splenocytes without and with 100 μM SNAP to obtain donor-specific Treg cells. Following 5-day culture, CD4⁺CD25^{high} cells were isolated by cell sorting and 2-5×10⁵ injected to SCID recipients transplanted with C57BL/6 skin allografts. Within 7 days all recipients were injected with 1×10⁵ CD4⁺CD45⁺ cells. The results revealed that recipients injected just with CD4⁺CD45⁺ cells all acutely rejected skin allografts (FIGS. 5C and 5D). In contrast, prior injection of CD4⁺CD25^{high} cells cultured without or with SNAP always induced long-term skin allograft survivals (FIGS. 5C and 5D). These results showed that NO-induced Treg cells are potent in vivo to prevent rejection and may be used for expansion of Treg population.

NO-Promotes Switching of CD4⁺CD25⁻ Cells into CD4⁺CD25⁺CD127⁻ Cells

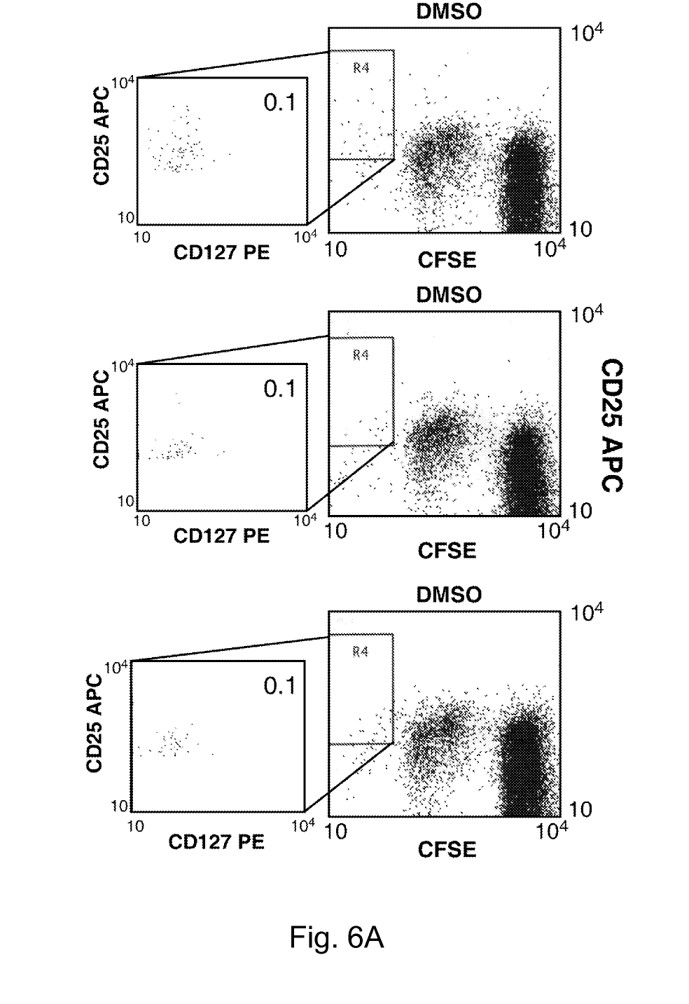

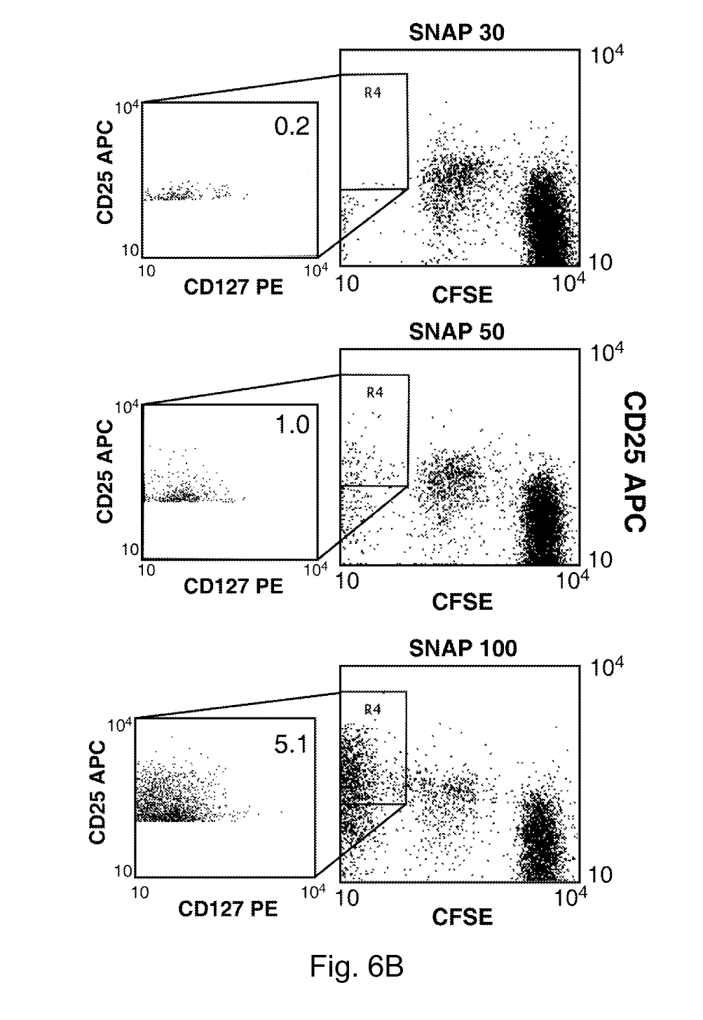

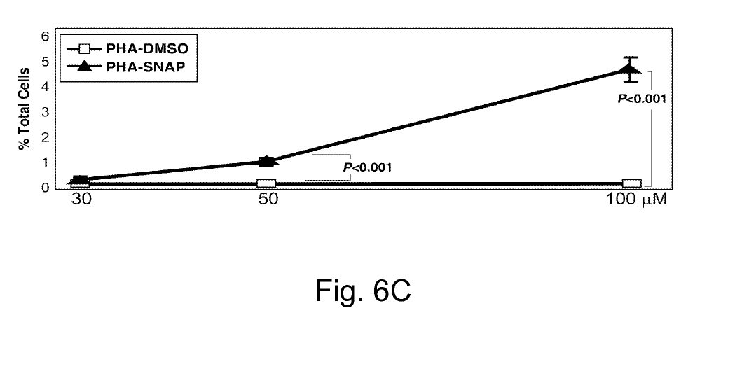

Experiments were performed to understand the mechanism by which NO increase the number of Treg cells. Isolated CD4⁺CD25⁻ cells labeled with carboxyfluorescein diacetate succinimidyl ester (CFSE) were co-cultured with CD4⁺CD25⁺ cells and stimulated with irradiated HLA-mismatched cells. Following 5 day-culture, CFSE⁺CD25^{high} cells were identified by FACS (FIG. 6). The results showed that NO increased switching of CD4⁺CD25⁻ cells into CD4⁺CD25⁺ regulatory T (Tr1) cells. Thus, NO promotes switching of naive CD4⁺ cells into next generation of Tr1 population.

### No-Releasing Agents Promote Generation of Treg Cells

In addition to S-Nitroso-N-acetyl-DL-penicillamine (SNAP) or sodium nitroprusside (SNP) other agents generate nitric oxide (NO) and these agents may also increase generation of Treg cells. For example, 3-morpholynosydnonimine (SIN-1, Naproxen (HCT-3012 [(S)-6-Methoxy-α-methyl-2-naphthaleneacetic Acid 4-(Nitrooxy)butyl Ester]), Sodium Nitroprusside, 2-2-(hydroxynitrosohydrazino)bis-ethamine (NOC-18), arginine, and others. To prevent apoptosis of Treg cells during culture in the presence of NO-releasing agents, phorbol 12-myristate 13-acetate (PMA) will be used to increase the generation of Treg cells. This agent prevents NO-induced apoptosis.

### REFERENCES

Sakaguchi S. Naturally arising CD4 regulatory T cells for immunologic self-tolerance and negative control of immune responses. Annu Rev Immunol. 2004;22: 531-562.
Gavin M, Rudensky A. Control of immune homeostasis by naturally arising regulatory CD4+ T cells. Curr Opin Immunol. 2003;15: 690-696.
Suri-Payer E, Amar A Z, Thornton A M, Shevach E M. CD4+CD25+ T cells inhibit both the induction and effector function of autoreactive T cells and represent a unique lineage of immunoregulatory cells. J Immunol. 1998;160: 1212-1218.
Roncarolo, M. G. & Levings, M. K. The role of different subsets of T regulatory cells in controlling autoimmunity. Curr. Opin. Immunol. 12, 676-683 (2000).
Shevach E M. Regulatory T cells in autoimmmunity. Annu Rev Immunol. 2000;18: 423-449.
Hall B M, Fava L, Chen J, et al. Anti-CD4 monoclonal antibody-induced tolerance to MHC-incompatible cardiac allografts maintained by CD4+ suppressor T cells that are not dependent upon IL-4. J Immunol. 1998; 161: 5147-5156.
Wood K J, Sakaguchi S. Regulatory T cells in transplantation tolerance. Nat Rev Immunol. 2003;3: 199-210.
Peng L, Kjaergaard J, Plautz G E, et al. Tumor-induced L-selectin high suppressor T cells mediate potent effector T cell blockade and cause failure of otherwise curative adoptive immunotherapy. J Immunol. 2002; 169: 4811-4821.
Gallimore A, Sakaguchi S. Regulation of tumour immunity by CD25+ T cells. Immunology. 2002; 107: 5-9.
Belkaid Y, Piccirillo C A, Mendez S, Shevach E M, Sacks D L. CD4+CD25+ regulatory T cells control Leishmania major persistence and immunity. Nature. 2002;420: 502-507.
Shevach, E. M. S. M. R., Piccirillo, C. A. & Thornton, A. M. Control of T-cell activation by CD4+CD25+ suppressor T cells. Immunol. Rev. 182, 58-67 (2001) |Article|
Roncarolo, M. G., Bacchetta, R., Bordignon, C., Narula, S. & Levings, M. K. Type 1 regulatory cells. Immunol. Rev. 182, 68-79 (2001)
Suvas S, Kumaraguru U, Pack C D, Lee S, Rouse B T. CD4+CD25+ T cells regulate virus-specific primary and memory CD8+ T cell responses. J Exp Med. 2003;198: 889-901.
Apostolou I, Sarukhan A, Klein L, von Boehmer H. Origin of regulatory T cells with known specificity for antigen. Nat Immunol. 2002;3: 756-763.
Kemper C, Chan A C, Green J M, Brett K A, Murphy K M, Atkinson J P. Activation of human CD4+ cells with CD3 and CD46 induces a T-regulatory cell 1 phenotype. Nature. 2003 Jan 23;421(6921):388-92.
Hogan M, Cerami A, Bucala R. Advanced glycosylation endproducts block the antiproliferative effect of nitric oxide. Role in the vascular and renal complications of diabetes mellitus. J Clin Invest. 1992 Sep;90(3):1110-5.
Koenen H J, Fasse E, Joosten I. CD27/CFSE-based ex vivo selection of highly suppressive alloantigen-specific human regulatory T cells. J Immunol. 2005 Jun 15;174(12):7573-83.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. The disclosures of all patents, patent applications and publications cited herein are hereby incorporated herein by reference, to the extent that they provide exemplary, procedural or other details supplementary to those set forth herein.

What is claimed is:

1. An ex vivo method of expanding a population of regulatory T-cells, comprising:
culturing a starting population of cells containing CD4⁺CD25⁻ T-cells in a growth medium;
introducing nitric oxide into the growth medium sufficient to potentiate switching of said CD4⁺CD25⁻ T-cells to CD4⁺CD25⁺ regulatory T-cells (Treg cells), whereby a subpopulation of Treg cells is produced;
allowing said Treg cells to proliferate in culture, to provide a final population of T-cells containing a greater number of Treg cells than did said starting population of T-cells.

2. The method of claim 1 further comprising isolating the resulting expanded population of Treg cells.

3. The method of claim 1, wherein said starting population comprises peripheral blood lymphocytes and said growth medium contains PHA.

4. The method of claim 1, wherein said nitric oxide is introduced into the growth medium by addition to the medium of a nitric oxide producing agent selected from the group consisting of S-Nitroso-N-acetyl-DL-penicillamine (SNAP), sodium nitroprusside (SNP), 3-morphylynosydnonimine (SIN-1), naproxen (HCT-3012 [(S)-6-Methoxy-α-methyl-2-naphthaleneacetic acid 4-(Nitrooxy)butyl ester]), sodium nitroprusside, 2-2-(hydroxynitrosohydrazino)bis-ethamine (NOC-18) and arginine.

5. The method of claim 1, wherein the concentration of nitric oxide after introduction of said NO producing agent is in the range of 10 to 100 micromolar.

6. An in vitro method of inhibiting proliferation of immune responsive T-cells, comprising culturing a population of said immune responsive T-cells in the presence of an immunogenic agent and in the presence of a population of Treg cells obtained in accordance with the method of claim 1.

7. A method of deterring or decreasing an undesired T-cell mediated immune response, comprising:
obtaining an isolated population of Treg cells produced according to the method of claim 1; and
transplanting said Treg cells into a host at the site of a potential or existing undesired immune response, whereby said undesired T-cell mediated immune response at said site is deterred or decreased.

8. The method of claim 7 wherein the resulting transplanted Treg cells block the undesired T-cell mediated immune response at the transplantation site.

9. The method of claim 7, wherein said site comprises an allograft.

10. The method of claim 9, wherein the resulting transplanted Treg cells enhance allograft acceptance at the transplantation site.

11. The method of claim 7, further comprising expanding the population of the resulting Treg cells at the transplantation site sufficient to inhibit chronic allograft rejection in the host.

12. The method of claim 7, further comprising expanding the population of the resulting Treg cells at the transplantation site sufficient to induce transplantation tolerance in the host.

13. The method of claim 7, wherein said host suffers from an autoimmune disease and said site comprises an existing or potential target site for attack by autoimmune T-cells.
